(19) United States
(12) Patent Application Publication
(10) Pub. No.: US 2017/0215704 A1
(43) Pub. Date: Aug. 3, 2017

(54) INSERTION DEVICE INCLUDING TRACTION MECHANISM

(71) Applicant: OLYMPUS CORPORATION, Tokyo (JP)

(72) Inventor: Masayo TSUMARU, Sagamihara-shi (JP)

(73) Assignee: OLYMPUS CORPORATION, Tokyo (JP)

(21) Appl. No.: 15/490,953

(22) Filed: Apr. 19, 2017

Related U.S. Application Data
(63) Continuation of application No. PCT/JP2015/079584, filed on Oct. 20, 2015.

(30) Foreign Application Priority Data
Oct. 28, 2014 (JP) 2014-219192

(51) Int. Cl. A61B 1/00; A61B 1/12; A61B 1/005; A61B 1/07

(52) U.S. Cl. CPC A61B 1/00128; A61B 1/00009; A61B 1/07; A61B 1/0008; A61B 1/126; A61B 1/00137; A61B 1/0057

(57) ABSTRACT

An insertion device includes an operation unit that is operable a traction member configured to apply a traction force to the operation unit, a channel member in which the traction member is inserted the channel member, a tubular seal member fixed to each of the channel member and the traction member and configured to deform along with a movement of the traction member and watertightly separate an interior of the channel member from a part of the traction member, a connecting member configured to clamp one end of the seal member with the part of the traction member, and a fixing member configured to fix the connecting member and the traction member in a state in which the seal member abutting against the connecting member and the traction member is pressed in the longitudinal axial direction.

### CROSS-REFERENCE TO RELATED APPLICATIONS

This is a Continuation Application of PCT Application No. PCT/JP2015/79584, filed Oct. 20, 2015, which was published under PCT Article 21(2) in Japanese.

This application is based upon and claims the benefit of priority from prior Japanese Patent Application No.2014-219192, filed Oct. 28, 2014 the entire contents of which are incorporated herein by references.

### BACKGROUND OF THE INVENTION

1. Field of the Invention

Embodiments described herein relate generally to an insertion device including a traction mechanism with sealing properties.

2. Description of the Related Art

In general, a treatment tool having various functions, for example, a forceps is inserted from an endoscope main body into the channel of an insertion portion and extended from an opening portion opened on a distal end side. In addition, a traction mechanism is known, which has an opening portion at a part of the distal end of the insertion portion on the peripheral surface side, and changes the extending direction of a guide wire or the like of the treatment tool extending from the opening portion in a desired direction. As a representative traction mechanism, a treatment tool raising base is known.

The treatment tool raising base can be made to pivot by manipulating the manipulation lever of a manipulation unit provided on the proximal end side of an endoscope, thereby bending the guide wire and directing the treatment tool to a direction desired by the operator. The manipulation lever and the treatment tool raising base are connected by a wire or the like inserted in a forceps raising channel.

Since the treatment tool raising base portion is inserted into a body cavity and used, a cleaning process is performed in the forceps raising channel during or after use. Hence, as proposed in, for example, patent literature 1: Jpn. Pat. Appln. KOKAI Publication No. 9-238898, an O-ring made of an elastic member is fitted on a wire or a wire holder to prevent a liquid such as a cleaning solution from entering from the opening portion into the manipulation unit of the endoscope via the channel, thereby implementing a structure with sealing properties by watertightness or airtightness.

It is an object of the present invention to provide an insertion device including a traction mechanism, which is mounted on a medical device, and is easy to assemble and implements high operability and reliable sealing properties without causing peel-off of a seal member.

### BRIEF SUMMARY OF THE INVENTION

According to an embodiment of the present invention, there is provided an insertion device comprising an operation unit that is operable and is provided in an insertion portion inserted into a lumen, a traction member configured to apply a traction force to operate the operation unit, a channel member in which the traction member is inserted such that the channel member is movable in a longitudinal axial direction of the traction member, a tubular seal member fixed to each of the channel member and the traction member and configured to deform along with a movement of the traction member and watertightly separate an interior of the channel member from a part of the traction member, a connecting member configured to clamp one end of the seal member with the part of the traction member, and a fixing member configured to fix the connecting member and the traction member in a state in which the seal member abutting against the connecting member and the traction member is pressed in the longitudinal axial direction.

Advantages of the invention will be set forth in the description which follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWINGS

The accompanying drawings, which are incorporated in and constitute a part of the specification, illustrate embodiments of the invention, and together with the general description given above and the detailed description of the embodiments given below, serve to explain the principles of the invention.

FIG. 1 is a view showing an example of the overall arrangement of an endoscope device that incorporates a traction mechanism in an endoscope main body as a medical device according to the present invention;

FIG. 2 is a view showing the conceptual arrangement of a raising base unit that is the traction mechanism incorporated in the endoscope main body;

FIG. 3A is a view showing the outer structure of the distal end portion of an insertion portion;

FIG. 3B is a view showing the outer structure of the distal end portion of the insertion portion;

FIG. 3C is a view showing the outer structure of the distal end portion of the insertion portion;

FIG. 4 is a view showing an example of the structure of the traction channel portion of the traction mechanism with sealing properties according to the first embodiment;

FIG. 5A is a view showing the structure of a section of the traction channel taken along a channel A-A in FIG. 4;

FIG. 5B is a view showing the structure of a section of the traction channel taken along a channel B-B;

FIG. 6 is a view showing an example of the structure of the traction channel portion of a traction mechanism with sealing properties according to the second embodiment;

FIG. 7A is a view showing a sectional structure including the distal end portion of a link member according to the embodiment;

FIG. 7B is a view showing a sectional structure including the distal end portion of the link member according to the embodiment;

FIG. 8 is a view showing an example of the structure of the traction channel portion of a traction mechanism with sealing properties according to the third and sixth embodiments;

FIG. 9 is a view showing an example of the structure of the traction channel portion of a traction mechanism with sealing properties according to the fourth and seventh embodiments;

FIG. 10 is a view showing an example of the structure of the traction channel portion of a traction mechanism with sealing properties according to the fifth embodiment;

FIG. 11A is a sectional view showing the structure of a connecting portion in a fixing portion on a distal end side in the traction channel portion of a traction mechanism according to the eighth embodiment; and

FIG. 11B is a sectional view showing the structure of a connecting portion in a fixing portion on a rear end side.

### DETAILED DESCRIPTION OF THE INVENTION

The embodiments of the present invention will now be described in detail with reference to the accompanying drawings.

### First Embodiment

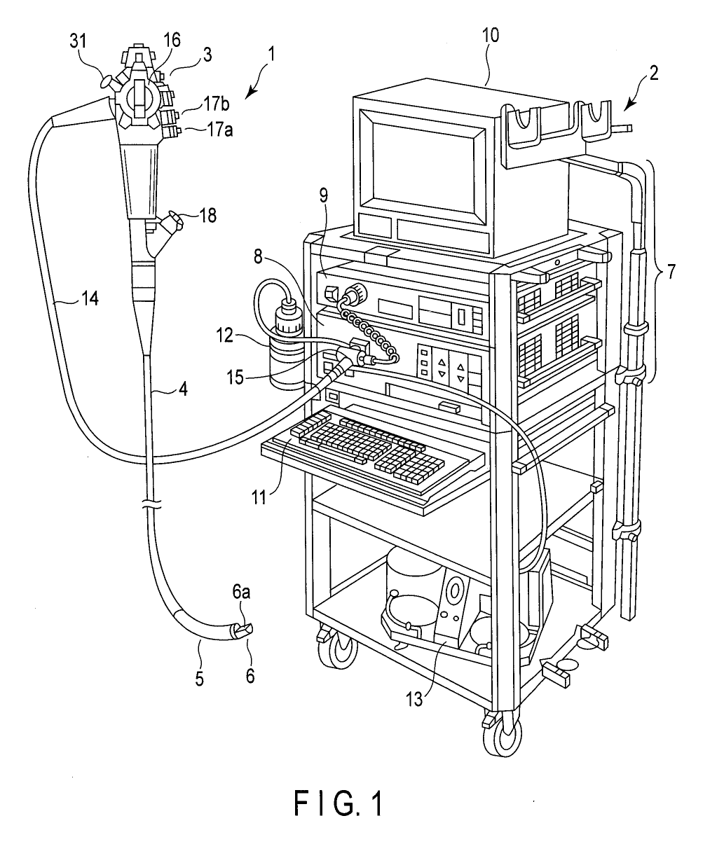

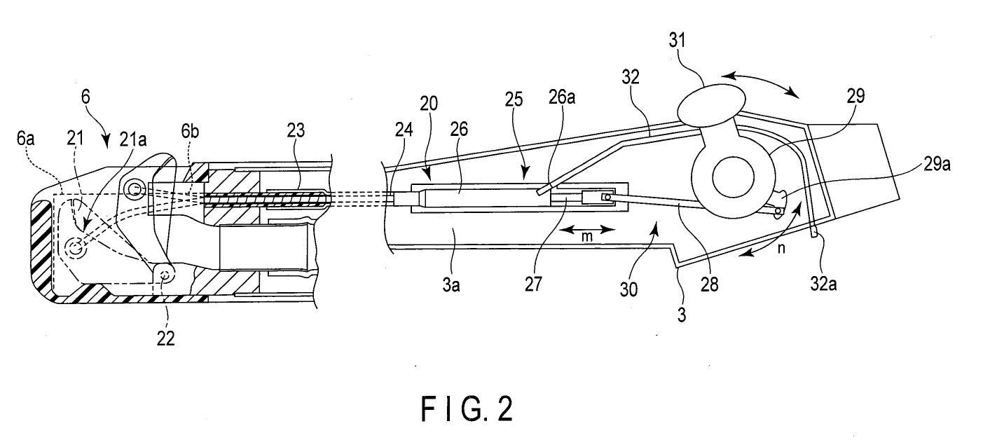

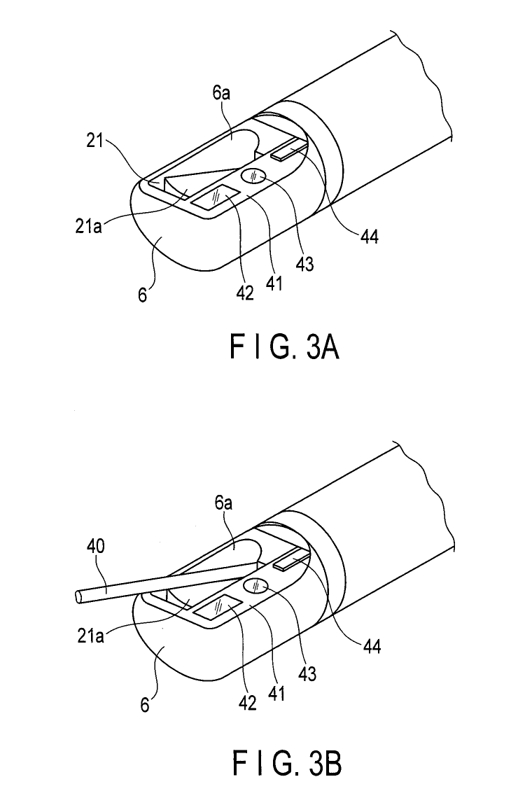

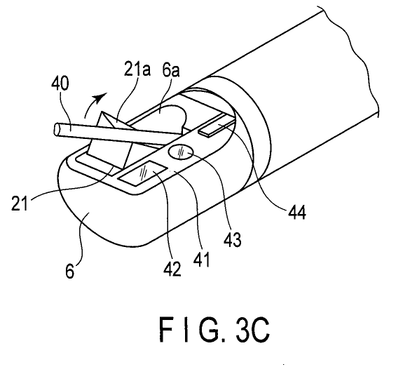

In this embodiment, an example will be described, in which using an endoscope that is a medical device as an example of an insertion device, a traction mechanism with sealing properties is applied to a raising base unit serving as an operation unit (driving mechanism) provided in an endoscope main body including an insertion portion and capable of operating so as to swing by traction. FIG. 1 is a view showing an example of the overall arrangement of an endoscope device that incorporates the traction mechanism in the endoscope main body (manipulation unit). FIG. 2 shows the conceptual arrangement of the raising base unit that is the operation unit made to swing by the traction mechanism incorporated in the endoscope main body. FIGS. 3A, 3B, and 3C show the outer structure of the distal end portion of the insertion portion.

In this embodiment, an example in which the traction mechanism is provided in the manipulation unit and the insertion portion of the endoscope device and applied to the raising base unit serving as the operation unit (driving mechanism) driven by traction will be described. However, the present invention is not limited to this. The traction mechanism can be arranged in the manipulation unit and easily applied to a pivot mechanism that is arranged as an operation unit (driving mechanism) operated by traction at the distal end of the insertion portion or another device that uses the channel of the insertion portion.

The endoscope device according to this embodiment is roughly divided into an endoscope main body (insertion device) 1 and endoscope equipment 7 serving as various kinds of external devices mounted on a movable trolley 2. In the following explanation, a flexible scope will be described as an example. However, a rigid scope can also be mounted in a similar manner.

The endoscope main body 1 includes an insertion portion (flexible tube) 4 to be inserted into a lumen such as a body cavity as an observation target, a bending portion 5 and a distal end portion 6 provided on the distal end side of the insertion portion 4, and an endoscope manipulation unit 3 that is provided on the proximal end side and bends the bending portion 5. In the following explanation, the side of the bending portion 5 will be referred to as a distal end side, and the side of the endoscope manipulation unit 3 will be referred to as a proximal end side.

The endoscope equipment 7 includes a light source device 8 that generates illumination light to irradiate an observation target portion, a video processor 9 that performs predetermined image processing for a captured video signal, a monitor 10 that displays a video signal as an observation image, and a keyboard 11 serving as an input unit.

An air supply pump or a suction unit 13 may be provided as the endoscope equipment 7. In addition, a bottle 12 that stores a liquid (for example, a physiological salt solution) to be used in cleaning of an observation window 43 (to be described later) provided at the distal end portion 6 is detachably attached to the trolley 2.

The endoscope main body 1 and the light source device 8 are connected by a connector 15 via a universal cable 14. The universal cable 14 includes a plurality of signal lines configured to transmit a video signal and the like, and gas and liquid supply paths (air and liquid supply channels) and discharge paths made of tubes as well as a light guide made of an optical fiber.

The connector 15 connected to the universal cable 14 on the side of the endoscope equipment 7 is branched to the signal lines, the tubes, and the light guide and connected to the devices. The endoscope manipulation unit 3 is provided with a bending manipulation unit 16 configured to bend the bending portion 5 in, for example, vertical and horizontal directions perpendicular to each other with respect to the insertion direction, an air supply/water supply button 17a, a suction operation button 17b, and a raising base manipulation lever (to be referred to as a manipulation lever hereinafter) 31 (to be described later). An insertion opening portion 18 of a forceps channel extending from a channel opening portion 6a through the insertion portion 4 is disposed between the endoscope manipulation unit 3 and the proximal end side of the insertion portion 4.

The distal end portion 6 of the endoscope main body 1 is made to a hard material into a cylindrical shape. As shown in FIG. 3A, a part of the peripheral surface from the distal end is cut. A flat surface 41 is formed at a portion, and the remaining portion is opened as the channel opening portion 6a. An illumination window 42 used for irradiation of illumination light, the observation window 43 used to observe an observation target portion, and a nozzle 44 configured to eject a fluid are arranged on the flat surface 41. The illumination window 42 is formed from an optical lens and irradiates an observation target portion with illumination light guided from the light source device 8 via the optical fiber. An observation image that has passed through the observation window 43 may be guided to an image sensor (not shown) provided in the endoscope manipulation unit 3 via an optical fiber or the like. Alternatively, an image may be formed by an image sensor (not shown) arranged under the observation window 43 (objective lens) in the distal end portion 6. The image sensor is provided integrally with an image processing circuit board using, for example, a CCD or CMOS sensor, and generates a video signal from an observation image by photoelectric conversion, and outputs the image to the video processor 9.

The nozzle 44 sprays one of a gas (for example, air) and a liquid (for example, water stored in the bottle 12) or a fluid mixture thereof to the surfaces of the illumination window 42 and the observation window 43 to remove dirt on the windows. In this operation, when the above-described air supply/water supply button 17a is pressed, the gas and the liquid are selectively ejected from the nozzle 44. When the suction operation button 17b is pressed, a pump (not shown) is driven to make a suction force act from the channel opening portion 6a of the distal end portion 6 via the treatment tool insertion channel extending through the insertion portion 4 and sucks and recovers an unnecessary fluid or the like in the body cavity.

A raising base unit (operation unit) 20 including the watertight traction mechanism according to the first embodiment will be described with reference to FIGS. 2, 3A, 3B, and 3C.

The raising base unit 20 is roughly divided into a raising base manipulation mechanism 30, a traction channel portion (or raising channel) 25, a guide tube 23, and a raising base 21. The raising base manipulation mechanism 30 is stored in the endoscope manipulation unit 3 on the proximal end side of the endoscope main body 1. The raising base 21 is provided in the channel opening portion 6a opened at the distal end portion 6. The guide tube 23 is inserted from the proximal end side of the insertion portion 4 up to the distal end portion 6. A raising wire (traction member) 24 that connects the raising base manipulation mechanism 30 and the raising base 21 and gives a traction force to operate the raising base unit 20 serving as an operation unit is disposed in the tube. The raising base 21 is pivotally provided in the channel opening portion 6a opened at the distal end portion 6.

As shown in FIGS. 2 and 3C, a surface 21a of the raising base 21 has a concave shape to easily engage with a treatment tool such as a guide wire to be bent. The raising base 21 is pivotally supported by a support member with a pivot point 22 formed in the bottom portion of the channel opening portion 6a. One end of the raising wire 24 is connected to a middle point of the raising base 21. The other end of the raising wire 24 is connected to the traction channel portion 25 including the traction mechanism.

The traction channel portion (channel member) 25 has sealing properties by watertightness (or airtightness) by a seal member 37 to be described later. The traction channel portion 25 includes a rod-shaped link member (traction member) 27 made of a metal material or the like to which the other end of the raising wire 24 is connected, a wire holding member 34, and a tubular outer member (traction channel member) 26 that covers the link member 27.

The raising base manipulation mechanism 30 includes a link member (link mechanism) 28 and the manipulation lever 31 pivotally supported in the endoscope manipulation unit 3 and having a knob exposed to the outside.

In the outer member 26, a traction mechanism having watertightness or airtightness according to each embodiment to be described later is provided for the link member 27 and the outer member 26. One end of the link member 28 is attached to the proximal end side of the link member 27 (link rod) extending from the outer member 26. A cleaning opening portion 26a is opened on the proximal end side of the outer member 26 to pour a cleaning solution from an oblique direction, and a cleaning tube 32 is connected. The housing of the endoscope manipulation unit 3 is provided with a cleaning base 32a configured to detachably connect the cleaning tube 32 and an external cleaning solution supply tube (not shown).

The manipulation lever 31 is provided with a projecting portion 29a that pivotally supports the other end of the link member 28. The link member 28 connects the manipulation lever 31 to the link member 27 to form a link mechanism. The link member 27 moves in the reciprocal moving (back and forth) direction in the longitudinal axial direction of the insertion portion in accordance with a manipulation on the manipulation lever 31. That is, when the link member 27 is moved, the raising wire 24 integrally moves, and the raising base 21 pivots.

As shown in FIG. 3B, a treatment tool (for example, a guide wire 40) inserted from the insertion opening portion 18 of the forceps channel extends from the channel opening portion 6a. The guide wire 40 passes on the surface 21a of the raising base 21. At this time, if the manipulation lever 31 is manipulated to pull the raising wire 24, the raising base 21 pivots about the pivot point 22 so as to rise, as indicated by a dotted channel in FIG. 2. At this time, the raising base 21 rises in a state in which the guide wire 40 is guided by the concave bottom portion of the surface 21a and bends the guide wire 40, as shown in FIG. 3C.

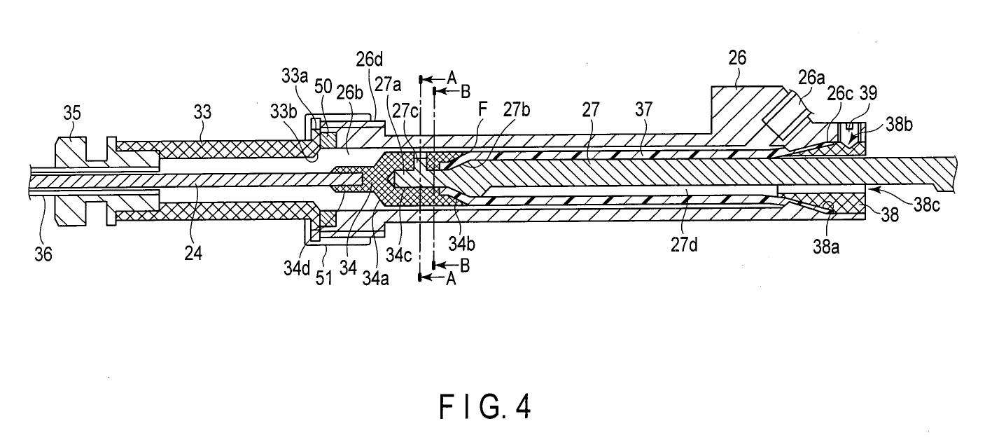

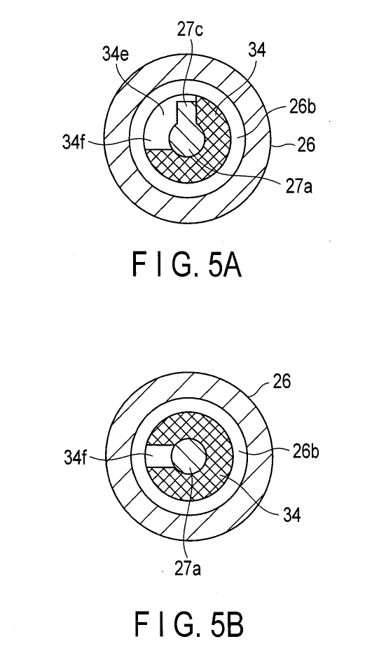

FIG. 4 is a view showing an example of the structure of the traction channel portion 25 of the traction mechanism with sealing properties according to the first embodiment. FIG. 5A shows the structure of a section of the traction channel taken along a channel A-A in FIG. 4. FIG. 5B shows the structure of a section of the traction channel taken along a channel B-B.

The traction mechanism is formed from the manipulation lever 31 and the traction channel portion 25. The traction channel portion 25 shown in FIG. 4 is an example of application to the raising base unit, and shows a state in which the raising base rises. Note that in the following explanation of each embodiment, the raising base side will be defined as a distal end portion, and the manipulation lever side will be defined as a proximal end portion.

The traction channel portion 25 is formed to have sealing properties mainly by the outer member 26, a distal end side outer member 33, a channel distal end portion 35, the wire holding member (connecting member) 34, the link member (second traction member) 27, and the tubular seal member 37. The outer member 26 forms a channel. Its outer shape is not particularly limited, but a circular shape is preferable as the sectional shape of the channel. Except the seal member 37, these members may be made of a metal material such as stainless steel or a stainless alloy, or another hard material, for example, a hard resin having the same rigidity as the metal material. Note that these members need to be made of materials capable of connection and working by welding, deposition, soldering, or an adhesive. The wire holding member 34 is a connecting member that integrally connects the raising wire 24 (first traction member) and the link member 27. Connection between the raising wire 24 and the wire holding member 34 may be done by an adhesive. If the wire holding member 34 is made of a metal, it may be fixed by soldering, squeezing, or caulking.

The outer member 26 has a tubular shape with two open ends. A clamp surface 26c [fourth clamp surface] formed from a tapered surface that increases the diameter of the opening on the proximal end side toward the outside is formed. The cleaning solution opening portion 26a that opens obliquely upward and forms an oblique flow from the periphery toward the center is provided on the proximal end side of the outer member 26. The cleaning solution opening portion 26a includes a connection base configured to connect the cleaning tube 32.

On the outer surface of the outer member 26 at the distal end, a notch portion to fit a waterproof packing 50 is formed, and a male screw 26d to be connected to the distal end side outer member (channel member) 33 is also formed.

The inner diameter of the distal end side outer member 33 is smaller than that of the outer member 26. On the proximal end side (the side connected to the outer member 26), a flange portion 33a that comes into contact with the waterproof packing 50 is formed. A tapered surface 33b configured to smoothen the flow of the cleaning solution in a portion 26b where the cleaning solution (to be described later) flows is formed on the inner surface side of the flange portion 33a.

A step is formed inside the distal end side outer member 33 on the distal end side, and the channel distal end portion 35 is fitted. The channel distal end portion 35 has a through hole opening at the center. An end portion of a pipe (channel member) 36 configured to insert the raising wire 24 is fixed in the opening. The distal end side outer member 33 is screwed to the outer member 26 by a ring-shaped fixing member 51 by making the flange portion 33a abut against the distal end portion of the outer member 26 such that watertightness is obtained by the waterproof packing 50.

Note that the traction members 24 and 27 are inserted in the channel members 26, 33, and 36 so as to be movable in the longitudinal axial diction of the traction members 24 and 27. The rod-shaped link member 27 loaded in the outer member 26 is restricted by a clamp surface 27b [first clamp surface] formed from a tapered surface tapered to the distal end side to forma distal end portion 27a of a small diameter. A projecting portion 27c (fixing member) functioning as a key tooth is provided at a part of the distal end portion 27a. The entire distal end portion forms a key shape. In this embodiment, a narrow groove along the longitudinal axial direction is formed in the outer surface of the link member 27 and caused to function as an air circulation path 27d that communicates with the outside air and allows supply/discharge of the air from/to the outside. The air circulation path 27d prevents a load generated by a pressure change caused by expansion and compression of air according to the reciprocal movement of the link member 27 or expansion and contraction of the seal member 37 and expansion and contraction of the seal member 37.

The wire holding member 34 includes, on the distal end side, a fixing portion 34d that fixes the raising wire 24 configured to raise the raising base 21. A tapered surface 34a configured to spread the cleaning solution to an entire necessary portion by smoothening a change in the sectional area of the portion 26b where the cleaning solution flows is formed on the rear side of the fixing portion 34d. The fluid supplied from the cleaning solution opening portion 26a thus circulates between the inner surface of the outer member 26 and the outer surface of the seal member 37. Although not illustrated, a stopper that stops the movement of the link member 27 is provided.

A clamp surface 34b [second clamp surface] formed from a tapered surface that is tapered inward is formed on the proximal end side of the wire holding member 34. A closed-end hole (insertion hole) 34c configured to fit the distal end portion 27a is formed at the center of the clamp surface 34b. In the closed-end hole 34c, an insertion groove (lock hole) 34f is formed in the axial direction (the horizontal direction in FIG. 4) of the hole to fit the projecting portion 27c as well. As shown in FIG. 5A, a window portion (lock hole) 34e that opens within an angular range of about 90° with respect to the peripheral surface is formed at the fixing portion. That is, the closed-end hole (insertion hole) 34c extending in the longitudinal axial direction on the surface on the side facing the link member 27 and the lock holes 34e and 34f opening from the closed-end hole 34c toward the outer surface in the radial direction are formed in the wire holding member (connecting member) 34.

The projecting portion 27c is fitted up to the position of the lock hole 34e through the lock hole 34f, and when reaches the lock hole 34e, pivots by about 90° to engage the projecting portion 27c with the lock hole 34e. That is, on the distal end side of the link member 27, a distal end portion capable of fitting in the closed-end hole (insertion hole) 34c is provided. When the distal end portion is fitted in the closed-end hole 34c, the projecting portion (fixing member) 27c and the lock holes 34e and 34f engage with each other, thereby fixing the wire holding member (connecting member) 34 and the link member 27.

In addition, the projecting portion (fixing member) 27c fixes the wire holding member 34 and the link member 27 so that the seal member 37 that abuts against the wire holding member (connecting member) 34 and the link member (traction member) 27 is pressed in the longitudinal axial direction. With this engaging, the link member 27 and the wire holding member 34 are integrally connected. Note that the angle range of pivot is not limited to about 90°, and is appropriately set. After the connection, an adhesive or a filler is applied to close the lock hole 34e, thereby fixing the projecting portion 27c to the wire holding member 34. The lock hole 34e is preferably completely filled and processed such that the surface becomes flush with the outer surface of the wire holding member 34.

The seal member 37 is made of an elastic material such as rubber, synthetic rubber, or resin to be deformable. In this embodiment, silicone rubber is employed as an example. The seal member 37 has a hollow tubular shape, preferably, a cylindrical shape. The distal end side is restricted so as to match the clamp surface 27b of the link member 27. The proximal end side has a spreading shape so as to match the clamp surface 26c of the outer member 26. For example, the seal member 37 can also be formed by molding using the link member 27 as a mold. Insert molding can also be applied. Note that the thickness (to be referred to as a wall thickness hereinafter) and strength of the seal member 37 according to this embodiment are set not cause permeation or infiltration of a liquid or a gas, and the thickness of the expanding/contracting portion is set within a range not to cause damage such as a break upon expansion and contraction.

A guide member 38 is fitted in the opening of the outer member 26 on the proximal end side and fixed by a screw. A through hole 38c configured to movably insert the link member 27 is formed at the center of the guide member 38. In addition, a surface that abuts against the clamp surface 26c of the opening on the proximal end side is formed into a clamp surface 38a [third clamp surface] formed from a tapered surface like the clamp surface 26c.

The guide member 38 is inserted into the outer member 26, thereby tightly pressing the seal member 37 against the outer member 26. A point contact member with small projections distributed on the entire surface may be adhered to the surface of the through hole provided in the guide member 38, which is in contact with the outer surface of the link member 27, thereby reducing the contact area between the link member 27 and the surface of the through hole and reducing the sliding resistance generated upon a movement in the axial direction. The through hole can be unclosed, as a matter of course. Since it is arranged in the manipulation unit, a lubricant such as grease may be used.

In addition, a concave portion 38b recessed in a wedge shape is formed in the outer surface of the guide member 38. A screw hole is formed in the outer surface of the outer member 26 on the proximal end side at a position where the concave portion 38b is exposed when the guide member 38 is fitted, and a projecting fixing screw 39 is screwed to the point. When the fixing screw 39 is fixed, the seal member 37 is pressed and fixed while being sandwiched between the outer member 26 and the guide member 38. The wire holding member 34 and the link member 27 are thus incorporated in the outer member 26.

FIG. 4 shows the seal member 37 having a natural length in a state in which the raising wire 24 is pulled by manipulating the manipulation lever 31, and the raising base 21 shown in FIG. 2 rises. In this embodiment, in a state (fallen state) in which the raising base 21 does not rise, the seal member 37 expands. The seal member 37 deforms in accordance with the movement of the raising wire 24 and the link member 27, that is, the traction member. According to the attachment structure of the seal member 37, neither creases nor folds are generated in the seal member 37 irrespective of traction or non-traction of the raising wire 24. The cleaning solution is poured obliquely from outside of the outer member 26 to inside via the cleaning solution opening portion 26a, flows between the inner surface of the outer member 26 and the outer surface of the seal member 37, passes through the pipe 36 of the channel distal end portion 35, flows out to the forceps channel, and is discharged from the channel opening portion 6a to outside.

According to the traction channel portion of this embodiment, no special member configured to fix the seal member is needed. In the manufacturing process of assembling the main members of the traction channel portion 25, one end (distal end side) of the seal member 37 is gripped (abuts against) the wire holding member 34 and the link member 27. The wire holding member 34 and the link member 27 are made close in the longitudinal direction (the interval in the longitudinal direction is decreased), and the seal member 37 is pressed in the longitudinal axial direction and fixed, thereby clamping one end of the seal member 37. That is, the link member 27 is fitted in the wire holding member 34 in a state in which the distal end portion 27a of the link member 27 is extended so as to be exposed from the opening portion of the seal member 37 having a restricted shape on the distal end side. One end of the seal member 37 is thus fixed in a state in which it is clamped between the clamp surface 27b and the clamp surface 34b.

When the guide member 38 is inserted into the outer member 26, the other end (read end side) of the seal member 37 is fixed while being clamped between the clamp surface 26c of the outer member 26 and the clamp surface 38a of the guide member 38 to be pressed in the longitudinal axial direction.

Hence, a sealing structure can be formed by sandwiching one end of the cylindrical seal member between the wire holding member and the link member and sandwiching the other end between the outer member and the guide member, that is, by fixing both ends by mechanical clamp. It is therefore possible to fix the seal member by a uniform mechanical clamp force (fixing force) irrespective of the uneven thickness or a dhesion strength difference of an adhesive layer in the fixing means using an adhesive. That is, one end side of the tubular seal member 37 is fixed between the wire holding member (connecting member) 34 and the link member (second traction member) 27, and the other end side is fixed to the outer member (channel member) 26 so that the seal member 37 covers the link member 27. The seal member 37 expands/contracts along with a movement of the link member 27 and separates the interior of the outer member 26 from the link member 27 to hermetically seal the interior of the seal member 37. In other words, it is possible to watertightly separate the interior of the outer member (channel member) 26 from a part of the link member (traction member) 27.

Hence, according to this embodiment, it is possible to provide an insertion device including a traction mechanism, which is mounted on a medical device, has a simple structure, and implements high operability and reliable sealing properties without causing peel-off of a seal member.

In this embodiment, an example in which an adhesive is used as an auxiliary is also proposed. Even a seal member that is hard to adhere by an adhesive is substantially mechanically fixed. Hence, the problem of peel-off of the seal member caused by fixing using only an adhesive does not arise. Note that the fixing force is a force necessary to extract the clamped seal member from the clamp surface. In addition, neither creases nor folds are generated on the surface of the seal member and close the channel. That is, since the fluid for cleaning can flow to the distal end side without any impediment of the flow in the channel, the detergency does not decrease.

### Second Embodiment

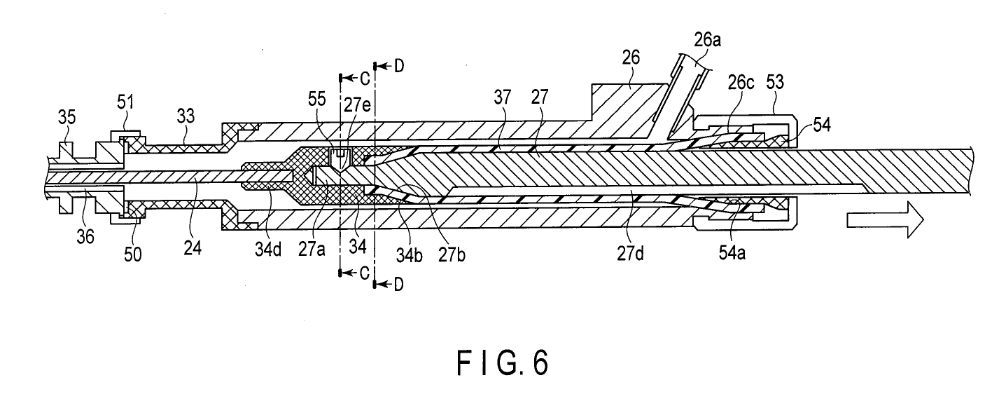

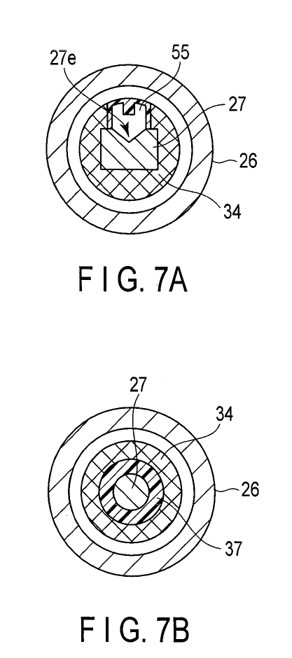

FIG. 6 is a view showing an example of the structure of a traction channel portion 25 of a traction mechanism with sealing properties according to the second embodiment. FIGS. 7A and 7B are views showing a sectional structure including the distal end portion of a link member according to this embodiment. The same reference numerals as in the above-described first embodiment denote the same constituent parts in this embodiment, and a detailed description thereof will be omitted.

In this embodiment, a channel distal end portion 35 and a distal end side outer member 33 are screwed by a fixing member 51 with intervention of an annular waterproof packing 50. The distal end side outer member 33 and an outer member 26 are fixed by an adhesive. If these members are made of a metal material, they can be connected using deposition or welding.

A distal end portion 27a of a link member 27 is formed to change from a circular section to a rectangular section, as shown in FIG. 7A. A concave portion 27e recessed in a wedge shape is formed in the upper surface of the rectangular shape. Since the seal member needs to be clamped by a uniform fixing force, the link member 27 has a circular section. A screw hole is formed in a wire holding member 34 at a position where the concave portion 27e is exposed when the link member 27 is fitted, and a projecting fixing screw 55 (fixing member) is fitted in the point. By the fixing screw 55, the link member 27 and the wire holding member 34 are fixed. The apex of the fixing screw 55 is formed to be located at a position lower than the outer surface of the wire holding member 34. The concave portion is preferably filled with an adhesive or the like to obtain a flat surface. When the distal end portion 27a is formed into a rectangular shape in this way, the concave portion 27e and the screw hole can easily be aligned when fitting the link member 27 in the wire holding member 34. Note that in each embodiment to be described below, a structure that implements fixing using a fixing screw is assumed to have the same structure as described above. Note that an adhesive may be used as an auxiliary as the fixing portion between the wire holding member 34 and the link member 27. The sectional shape of the distal end portion 27a is not limited to the rectangular shape and may be a circular shape including a long circular shape and an elliptical shape.

A through hole with a diameter that allows the link member 27 to reciprocally move is formed in an annular guide member 54. A clamp surface 54a facing a clamp surface 26c described above is formed on the outer surface. The guide member 54 is inserted from the proximal end side of the outer member 26. The guide member 54 and the outer member 26 clamp a seal member 37. In addition, a fixing member 53 is buried. By the fixing member 53, the guide member 54 is fixed to the outer member 26 in a state in which the guide member 54 is pressed into a wedge shape against the clamp surface 26c.

The distal end side of the seal member 37 according to this embodiment is fitted in a clamp surface 27b [first clamp surface] of the link member 27 and clamped by a clamp surface 34b [second clamp surface] of the wire holding member 34 and fixed. The proximal end side of the seal member 37 is clamped between the clamp surface 26c [third clamp surface] of the outer member 26 and the clamp surface 54a [fourth clamp surface] of the guide member and fixed.

According to this embodiment, it is possible to obtain the same effects as in the above-described first embodiment.

### Third Embodiment

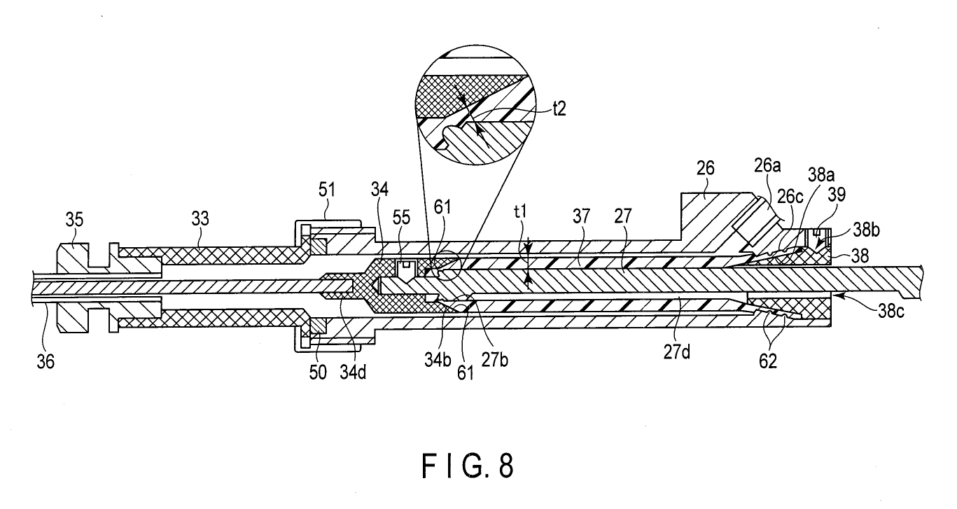

FIG. 8 is a view showing an example of the structure of a traction channel portion 25 of a traction mechanism with sealing properties according to the third embodiment. The same reference numerals as in the above-described first embodiment denote the same constituent parts in this embodiment, and a detailed description thereof will be omitted.

In this embodiment, at least one annular projecting portion 61 is formed on each of a clamp surface 27b of a link member 27 and a clamp surface 38a of a guide member 38. The projecting portion 61 has a semi-circular, semi-elliptic, semi-cylindrical, parabolic (conic), or rectangular section on the periphery of the clamp surface 27b at the time of manufacturing. Note that if the sectional shape is rectangular, the edge portions are preferably rounded.

When the link member 27 is fitted in a wire holding member 34, and a fixing screw 55 is screwed, the distal end side of the seal member 37 is clamped and fixed between a clamp surface 34b and the clamp surface 27b with the projecting portion 61. Similarly, when the link member 27 and the wire holding member 34 which are integrated are inserted in an outer member 26, the rear end side of the seal member 37 is clamped and fixed between a clamp surface 26c and the clamp surface 38a with a projecting portion 62.

According to this embodiment, it is possible to obtain the same effects as in the above-described first embodiment. Additionally, in this embodiment, at the projecting portion 61 provided on the clamp surface 27b of the link member 27 and the projecting portion 62 provided on the clamp surface 26c, a pressing force larger than at the other portions is locally channelarly applied. This increases the fixing force to fix the seal member 37 and makes it difficult for the seal member 37 to peel off from the link member 27 and the guide member 38.

Furthermore, in this embodiment, an example in which one projecting portion is formed on the seal surface has been described. However, the present invention is not limited to this, and the seal member can be fixed more firmly by arranging a plurality of (rows) of projecting portions. Note that in this embodiment, the seal member 37 having a sheet shape with a flat surface has been exemplified. However, a seal member may be molded using the link member 27 with the projecting portion 61 as a mold. If a concave portion corresponding to the projecting portion 61 is formed in the seal member 37, the concave portion engages with the projecting portion on the tapered surface upon clamping, and the disengagement is more difficult. The same effect can be obtained in the projecting portion 62 as well. The projection may be formed not on the link member 27 but on the wire holding member 34 or not on the outer member 26 but on the guide member 38.

### Fourth Embodiment

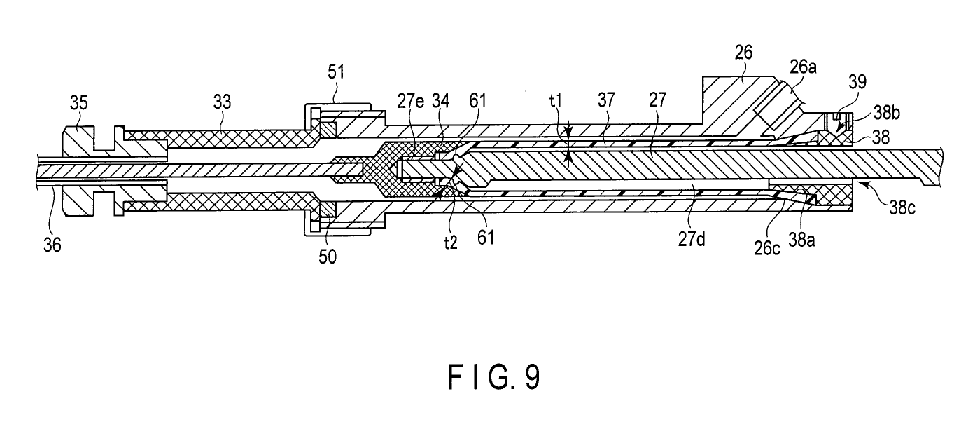

FIG. 9 is a view showing an example of the structure of a traction channel portion 25 of a traction mechanism with sealing properties according to the fourth embodiment. The same reference numerals as in the above-described first embodiment denote the same constituent parts in this embodiment, and a detailed description thereof will be omitted.

In this embodiment, at least one annular projecting portion 61 is formed on a clamp surface 27b of a link member 27. The projecting portion 61 has the same sectional shape as in the above-described third embodiment. A male screw 27e is formed at a distal end portion 27a of the link member 27.

A clamp surface 34b is formed on the proximal end side of a wire holding member 34, and a closed-end hole is formed at the center. A female screw is formed in the closed-end hole. The distal end portion 27a of the link member 27 is fitted and screwed in the hole. At this time, the distal end side of a seal member 37 is clamped and fixed between the clamp surface 27b and the clamp surface 34b.

The rear end side of the seal member 37 is clamped and fixed between a clamp surface 26c and clamp surface 38a, as in the above-described first embodiment. A guide member 38 is screwed by a fixing screw 39.

According to this embodiment, it is possible to obtain the same effects as in the above-described first embodiment. In addition, since the wire holding member 34 and the link member 27 are connected and fixed by screwing, the number of parts and the operation man-hour can be decreased as compared to a fixing method using the above-described fixing screw. Furthermore, since the inner diameter of the seal member 37 is larger than the outer diameter of the link member 27, the contact resistance between them can be reduced.

### Fifth Embodiment

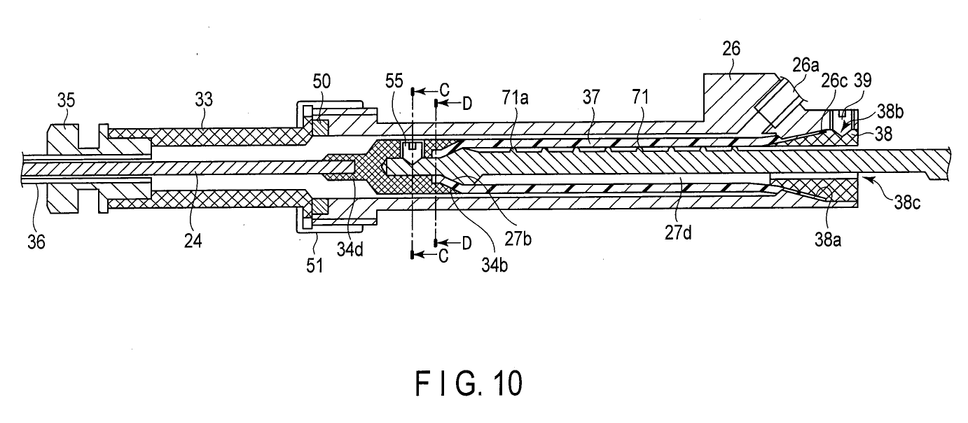

FIG. 10 is a view showing an example of the structure of a traction channel portion 25 of a traction mechanism with sealing properties according to the fifth embodiment. The same reference numerals as in the above-described first embodiment denote the same constituent parts in this embodiment, and a detailed description thereof will be omitted.

In this embodiment, the fixing method for a seal member 37 is the same as in the above-described third embodiment. The distal end side of the seal member 37 is clamped and fixed between a clamp surface 27b and a clamp surface 34b. The rear end side is clamped and fixed between a clamp surface 38a and a clamp surface 26c.

A link member 71 according to this embodiment has a plurality of annular projecting portions 71a that come into point or channelar contact with a portion facing a region where the seal member 37 expands/contracts.

When the projecting portions 71a are provided, the contact area between the link member 71 and the seal member 37 becomes small, and therefore, the contact resistance between them decreases. This can prevent the seal member 37 from adhering to the surface of the link member 71 and expanding/contracting together with the link member. Note that as for the shape of the projecting portion, a plurality of dot shapes (for example, semispherical or cylindrical shapes) or a wavy shape that waves in the longitudinal axial direction may be applied in addition to the annular projecting portion.

### Sixth Embodiment

An example of the structure of a traction channel portion 25 of a traction mechanism with sealing properties according to the sixth embodiment will be described with reference to FIG. 8. Note that the same reference numerals as in the above-described third embodiment denote the same constituent parts in this embodiment, and a detailed description thereof will be omitted.

In the example of the structure according to this embodiment, the wall thickness of a seal member 37 changes between a fixed portion and an expansion/contraction portion. A projecting portion 61 is provided on a clamp surface 27b of a link member 27 shown in FIG. 8. In this embodiment, a thickness t1 of the expansion/contraction region of the seal member 37 is made larger than a thickness t2 of the fixed region clamped between the two clamp surfaces (t1>t2). Alternatively, the thickness t2 of the fixed region is made smaller than the thickness t1 of the current expansion/contraction region.

According to this embodiment, the wall thickness of the fixed region is made smaller than the wall thickness of the expansion/contraction region, thereby improving the durability of the expansion/contraction region without increasing the wall thickness, that is, the outer diameter of the fixed region. If forming the projecting portion 61 on a clamp surface 38a of a guide member 38, as in the above-described third embodiment, is combined with this embodiment, the seal member 37 can be fixed without decreasing the fixing force.

### Seventh Embodiment

An example of the structure of a traction channel portion 25 of a traction mechanism with sealing properties according to the seventh embodiment will be described with reference to FIG. 9. Note that the same reference numerals as in the above-described fourth embodiment denote the same constituent parts in this embodiment, and a detailed description thereof will be omitted.

In the example of the structure according to this embodiment, the wall thickness of a seal member 37 changes between a fixed portion and an expansion/contraction portion. A projecting portion 61 is provided on a clamp surface 27b of a link member 27 shown in FIG. 9. In this embodiment, a thickness t1 of the expansion/contraction region of the seal member 37 is made smaller than a thickness t2 of the fixed region clamped between the two clamp surfaces (t1<t2). Alternatively, the thickness t2 of the fixed region is made larger than the thickness t1 of the current expansion/contraction region.

According to this embodiment, the wall thickness of the fixed region of the seal member 37 is made larger than the wall thickness of the expansion/contraction region. This can increase the strength of the fixed region in the clamped state to reduce a rupture or breakage, and can stabilize the crushing ratio by clamping. In addition, when the wall thickness of the expansion/contraction region decreases, the load on the manipulation of the manipulation lever is reduced.

### Eighth Embodiment

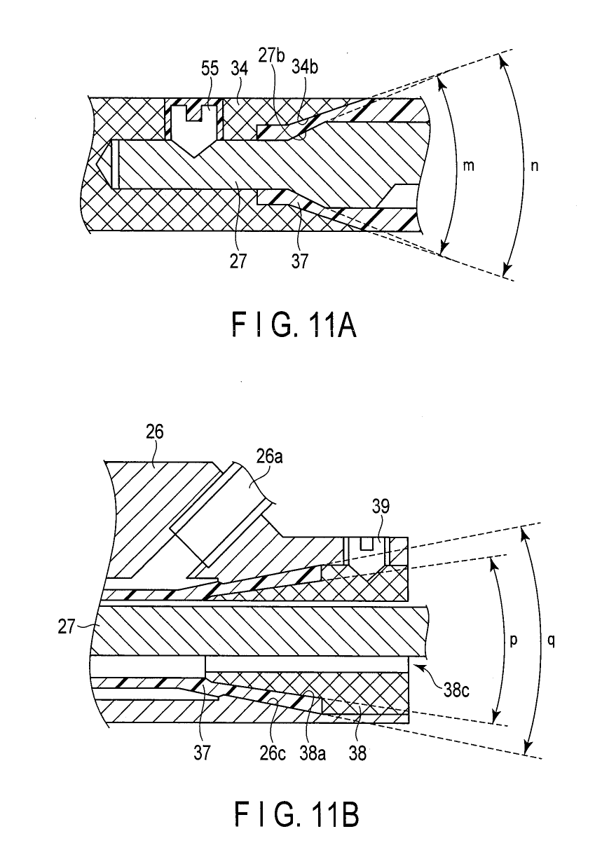

FIG. 11A is a view showing the structure of a section of the connecting portion between a link member 27 and a wire holding member 34, which is the fixing portion on the distal end side in the traction channel portion of a traction mechanism with sealing properties according to the eighth embodiment. FIG. 11B is a view showing the structure of a section of the connecting portion between a guide member 38 and an outer member 26, which is the fixing portion on the rear end side. Note that the same reference numerals as in the above-described first embodiment denote the same constituent parts in this embodiment, and a detailed description thereof will be omitted.

In this embodiment, let m be the taper angle between tapered surfaces of a clamp surface 27b [first clamp surface] on the distal end side, and n be the taper angle of a clamp surface 34b [second clamp surface]. In addition, let p be the taper angle of a clamp surface 38a [third clamp surface] on the proximal end side, and q be the taper angle of a clamp surface 26c [fourth clamp surface].

In this embodiment, the angles on the distal end side are set to m>n, and the angles on the proximal end side are set to p<q. That is, if the wall thickness of the fixed region of a seal member 37 does not change, the crushing ratio of the seal member rises toward the expansion/contraction region. Since the expansion/contraction region side is fixed more firmly, a fluid can be more reliably prevented from entering the fixed region in the clamped state.

In addition, the wire holding member 34 and the link member 27 are fixed in a state in which the seal member 37 covers the outer surface of the link member 27. At this time, at a portion (F in FIG. 4) where the wire holding member 34 and the link member 27 face each other, to prevent turbulence in the flow of a fluid, the wire holding member 34 and the seal member 37 covering the link member 27 are preferably formed into almost the same sectional shape to obtain smoothness as much as possible between the wire holding member 34 and the outer surface of the seal member 37 covering the link member 27. For example, there is a method of setting a value obtained by halving the difference between the outer diameter of the wire holding member 34 and the outer diameter of the link member 27 to the thickness of the seal member 37 at the connecting portion. Note that the gap between the outer surface of the wire holding member 34 and the outer surface of the seal member 37 may be filled with an adhesive that elastically solidifies.

As described above, an insertion device includes an operation unit that is operable and is provided in an insertion portion inserted into a lumen, a traction member configured to apply a traction force to operate the operation unit, a channel member in which the traction member is inserted such that the channel member is movable in a longitudinal axial direction of the traction member, a tubular seal member fixed to each of the channel member and the traction member and configured to deform along with a movement of the traction member and watertightly separate an interior of the channel member from a part of the traction member, a connecting member configured to clamp one end of the seal member with the part of the traction member, and a fixing member configured to fix the connecting member and the traction member in a state in which the seal member abutting against the connecting member and the traction member is pressed in the longitudinal axial direction.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An insertion device comprising:
an operation unit that is operable and is provided in an insertion portion inserted into a lumen;
a traction member configured to apply a traction force to operate the operation unit;
a channel member in which the traction member is inserted such that the channel member is movable in a longitudinal axial direction of the traction member;
a tubular seal member provided in the channel member and configured to cover a part of the traction member;
a connecting member configured to fix the traction member such that one end of the seal member is brought into tight contact with the traction member; and
a guide member configured to clamp other end of the seal member in a state in which the seal member is pressed in the longitudinal axial direction with the channel member so as to watertightly separate an interior of the channel member from the part of the traction member.

2. The device according to claim 1, wherein the seal member is fixed to the channel member so as to deform along with a movement of the traction member and watertightly separate the interior of the channel member from the part of the traction member.

3. The device according to claim 1, further comprising a fixing member configured to fix the connecting member to the traction member in a state in which one end of the seal member is clamped in the longitudinal axial direction between the traction member and the connecting member.

4. The device according to claim 3, wherein the traction member includes a first traction member connected to the operation unit, and a second traction member connected to the first traction member via the connecting member, and
the fixing member fixes the connecting member and the second traction member.

5. The device according to claim 4, wherein the first traction member includes a wire with one end connected to the operation unit, and
the connecting member is connected to the other end of the wire.

6. The device according to claim 4, wherein an outer surface of the second traction member includes a first clamp surface with a tapered surface,
an inner surface of the connecting member includes a second clamp surface with a tapered surface, and
one end side of the seal member is clamped and fixed between the first clamp surface and the second clamp surface when fixing the connecting member and the second traction member by the fixing member.

7. The device according to claim 1, wherein an outer surface of the guide member includes a third clamp surface with a tapered surface,
an inner surface of the channel member includes a fourth clamp surface with a tapered surface, and
other end side of the seal member is clamped and fixed between the third clamp surface and the fourth clamp surface when fixed by the guide member.

8. The device according to claim 4, wherein the connecting member includes an insertion hole extending in the longitudinal axial direction in a surface on a side facing the second traction member, and a lock hole opening from the insertion hole toward an outer surface in a radial direction, and
a distal end portion fittable in the insertion hole is provided on a distal end side of the second traction member, and when the distal end portion is fitted in the insertion hole, the fixing member engages with the lock hole to fix the connecting member and the second traction member.

9. The device according to claim 4, wherein the seal member has a tubular shape that covers the second traction member while fixing one end side between the connecting member and the second traction member and fixing other end side to the channel member, expands/contracts along with a movement of the second traction member, and separates the interior of the channel member from the second traction member to hermetically seal the interior of the channel member.

10. The device according to claim 6, wherein one of the first clamp surface and the second clamp surface includes an annular projecting portion formed along a radial direction of at least one peripheral surface.

11. The device according to claim 6, wherein the seal member is formed such that a wall thickness of a fixed region clamped between the first clamp surface and the second clamp surface is different from a wall thickness of an expansion/contraction region that expands/contracts along with a movement of the second traction member.

12. The device according to claim 4, wherein a groove serving as an air circulation path communicating with outside air is provided in an outer surface of the second traction member along a longitudinal direction of the second traction member.

13. The device according to claim 4, further comprising an opening portion configured to supply a fluid that circulates through the interior of the channel member,
wherein the seal member covers an outer surface of the second traction member,
the seal member is arranged inside the channel member, and
the fluid supplied from the opening portion circulates between an inner surface of the channel member and an outer surface of the seal member.

14. The device according to claim 4, wherein the seal member covers an outer surface of the second traction member, and
at a portion where the second traction member and the connecting member face each other, an outer surface of the connecting member and the outer surface of the second traction member covered with the seal member have substantially the same sectional shape.
